(19) United States
(12) Patent Application Publication
(10) Pub. No.: US 2023/0263849 A1
(43) Pub. Date: Aug. 24, 2023

(54) FUNCTIONALIZED CELLULOSE NANOCRYSTALS STABILIZED SMART PICKERING EMULSION FOR ENHANCED PROBIOTIC DELIVERY

(71) Applicant: UNIVERSITY OF WATERLOO, Waterloo (CA)

(72) Inventors: Michael Kam Chiu Tam, Waterloo (CA); Jiyoo Baek, Waterloo (CA); Mohankandhasamy Ramasamy, Waterloo (CA); Gun-Hee David Cho, Thornhill (CA)

(21) Appl. No.: 18/174,111

(22) Filed: Feb. 24, 2023

Related U.S. Application Data
(60) Provisional application No. 63/372,209, filed on Feb. 24, 2022.

(51) Int. Cl. A61K 36/064; A61K 9/113; A61K 47/12; A61K 47/38; A61K 47/36; A61K 47/02; C12Q 1/06

(52) U.S. Cl. CPC A61K 36/064; A61K 9/113; A61K 47/02; A61K 47/12; A61K 47/36; A61K 47/38; C12Q 1/06

(57) ABSTRACT

The efficacy in the use of probiotics is compromised due to their lack of survivability in gastric conditions (pH 1.5-3), leading to a large reduction in viable probiotic cells. The present disclosure describes cellulose nanocrystals (CNCs) modified with ferulic acid (FA) and shellac (SH) to produce two types of new coating materials, which are environment friendly and harmless to humans. These coating materials were developed and utilized to formulate Pickering emulsions (W₁/O/W₂ and W/O) for probiotic encapsulation. Based on their pKa values, greater than pKa, carboxyl groups from the CNCFA and CNCSH based Pickering emulsions were deprotonated, inducing phase separation and allow yeast releasing. This system could be further investigated for functionalized food-based applications to deliver active substances, such as probiotics, at high pH. Such delivery systems can be applied to human, animal, and aquatic systems.

### CROSS-REFERENCE TO RELATED APPLICATIONS

The present application claims priority to and the benefit of U.S. Provisional Application No. 63/372,209, filed 24 Feb. 2022 and titled FUNCTIONALIZED CELLULOSE NANOCRYSTALS STABILIZED SMART PICKERING EMULSION FOR ENHANCED PROBIOTIC DELIVERY, which is incorporated by reference herein in its entirety for all purposes.

### FIELD OF THE DISCLOSURE

The present disclosure relates to the modification and use of ferulic acid (FA) and shellac (SH) functionalized cellulose nanocrystals (CNC) (CNCFA and CNCSH) for probiotic delivery.

### BACKGROUND OF THE DISCLOSURE

Probiotics effectively inhibit harmful bacteria by secreting antibacterial bacteriocin, lactic acid, intestinal settlement of harmful bacteria, which enhances our immune system. Probiotics must survive by withstanding the gastric acid secreted in the stomach, bile from the gallbladder, and various digestive enzymes from the small intestines. However, adding probiotic cells directly to foods resulting in a significant reduction in cell viability during storage and passage through the intestinal tract. Therefore, probiotics must be encapsulated with another protective material, where encapsulation protects probiotic cells from external environmental factors and promotes bacterial viability during processing, storage and digestion.

Cellulose nanocrystals (CNC) are natural materials with emerging food and biomedical applications due to their excellent physical, chemical, and biological properties, including biocompatibility and biodegradability (Du et al., 2019; Kim et al., 2019). CNCs are produced by acid hydrolysis of cellulose fibers with length of between 100 to 250 nm. It is considered as one of the favorable candidates for Pickering emulsion preparation due to both the hydrophilic and hydrophobic edge plane, which has been successfully proven in the preparation of food emulsion systems.

Ferulic acid (FA) is present abundantly in plant cell walls and seeds of plants, which are phenolic compounds containing carboxylic acid groups (COOH), hydroxyl groups (—OH), and ethylenic bonds (C═C). FA is a powerful antioxidant that neutralizes free radicals and inhibits reactive oxygen species (ROS) or nitrogen to protect living organisms. The phenoxy and hydroxy groups of FA donate electrons to scavenge the free radicals. In addition, the FA’s benzene ring stops the free radical reaction, and the carboxylic group also attacks the free radical site and prevents premature oxidation of DNA and lipids as this group binds to the lipid bilayer. FA has many beneficial effects on our health, and food industries have been utilizing it as a preservative and an ingredient in sport food, such as ergogenic aid.

Shellac is a natural resin that is approved as a food-safe additive by the U.S Food and Drug Administration. This alkaline or alcohol soluble lac insect resin is hydrophobic, biodegradable, and renewable, making it a suitable candidate for commercial applications. The resin’s unique chemical structure comprises of hydrophilic aleuritic acid and hydrophobic cyclic terpenic acids, connected through ester bonds, making this an amphiphile. Additionally, this resin consists of other functional groups (—CHO, —OH, —COOH), giving it ample room to interact with various polymers or chemical components through electrostatic or hydrogen-bonding to form the desired systems. Thus, shellac is considered a suitable candidate to be used as a coating material for functional food systems.

### SUMMARY

In one aspect there are two provided different methods to produce ferulic Acid (FA) coated cellulose nanocrystals (CNC) and shellac (SH) complex with CNC upon the addition of CaCl₂ for encapsulating and preserving probiotics in acidic condition.

In any aspect or embodiment described herein, (I) The process for synthesizing CNCFA comprises, or is a derivative of the following:
dispersing CNCs in ethanol and dispersing FA in ethanol;
introducing or combining the FA ethanol solution into the CNC/ethanol dispersion;
stirring of CNCs and FA mixture for 24 hours;
air drying the CNCFA mixture in an oven for 24 hours;
redispersing the CNCFA nanoparticle in an optionally adjusted liquid suspension.

In another aspect, there is a provided a method to produce a modified starch nanoparticle (mSNP) as defined herein, or as a derivative of the following:
The dispersion of corn starch in DI water at 90° C. to prepare gelatinized form of starch nanoparticle;
Setting temperature at 70° C. and then adding ethanol dropwise under constant stirring for 2 hours;
Using centrifuge to remove water and washing the suspension with ethanol;
Allowing the modified starch nanoparticles to be dried in an oven for 24 hours;
The shearing and grounding of starch aggregates using external forces to generate nano-scale dispersions of SNP, which is categorized as mSNP.

In one aspect, there is a provided method for the generation of a water-in-oil (W/O) Pickering emulsion encapsulation for activated yeast and/or probiotic, or a derivative thereof comprised in the following procedure herein:
The preparation of an oil phase in which mSNP is dispersed at 2% or an optionally adjusted percentage by weight of vegetable oil (or a derivative thereof) used;
The preparation of a water phase characterized by an addition of an adjustable ratio of Baker’s yeast (or a derivative thereof) and glucose (C₆H₁₂O₆) into water;
The preparation of oil phase and water phase combined at a ratio respectively of 7:3; and
The homogenization of oil phase and water phase at high speed for an optionally adjustable time to produce W/O emulsions with hydrophobic mSNP as stabilizing emulsifiers at each emulsion’s phase interface.

In another aspect, there is an alternative method for the generation of a water-in-oil Pickering emulsion encapsulation for activated yeast, or a derivative thereof comprised of the following:
The preparation of an oil phase using vegetable oil or a derivative thereof;
The preparation of a water phase characterized by an addition of an adjustable ratio of Baker’s yeast (or a derivative thereof) and glucose (C₆H₁₂O₆) into water;
The preparation of a dispersed mSNP in water;
The preparation of oil, water phase, and mSMP solution at a ratio respectively of 14:5:1;
The homogenization of oil and water phase at high speed for an optionally adjusted time to create W/O emulsions;
The immediate injection of mSMP solution upon W/O emulsification to act as a stable emulsifier at each emulsion’s phase interface.

In one aspect, there is a method for the generation of a water-in-oil-in-water (W/O/W) multiple Pickering emulsion for the encapsulation of activated yeast, or a derivative thereof comprised in the following procedure herein:
The preparation of a water phase characterized by the dispersion of CNCFA into water at an optionally adjusted pH;
The preparation of a W/O phase, encapsulating activated Baker’s yeast (or a derivative thereof);
The homogenization of water phase with W/O phase at high speed for an optionally adjusted time to create W/O/W emulsions;
The intended use for CNCFA to act as the hydrophilic, stabilizing, emulsifier at the water and W/O interface;
The emulsions’ pH was transferred to 2 or 7.5 to mimic the gastric and intestinal pH conditions.

In any aspect or embodiment described herein, (II) This process for synthesizing CNCSHCA comprises, or is a derivative of the following protocol:
Dissolving SH in 100% denatured ethanol and redispersed in MQ water to achieve the desired concentration; and
CNCSHCA is prepared with shellac dispersion, CNC dispersion and CaCl₂ solution

In one aspect, there is a provided method for the generation of a water-in-oil (W/O) Pickering emulsion encapsulation for activated yeast, or a derivative thereof comprised of the following procedure herein:
The preparation of shellac dispersion as a water phase characterized by an addition of an adjustable ratio of Baker’s yeast (or a derivative thereof);
The preparation of oil phase and water phase combined at a ratio respectively of 6:4;
The homogenization of oil phase and water phase at high speed for an optionally adjustable time to create W/O emulsions;
Add calcofluor white dyed CNC (CNC-CW) and CaCl₂ solution; and
The emulsion’s pH was changed to 2 or 7.5 to mimic the gastric and intestinal pH conditions.

### BRIEF DESCRIPTION OF THE DRAWINGS

The present disclosure is illustrated with reference to the following drawings, in which:

FIGS. 1A and 1B. FIG. 1A shows particle size and zeta-potential of CNC from pH 1-9 and FIG. 1B shows particle size and zeta-potential of CNCFA from pH 1-9.

FIG. 2 is the chemical structure of CNCFA by hydrogen bonding.

FIGS. 3A, 3B, and 3C. FIG. 3A shows the influence of pH on maximum adsorption capacity of FA on CNC, FIG. 3B shows images of contact angles of native CNC and CNCFA, and FIG. 3C is a schematic illustration demonstrating the pKa of FA.

FIG. 4 is the free-radical scavenging activity of FA and CNCFA at pH 2 and 7.5 by DPPH assay.

FIGS. 5A, 5B, and 5C are TEM images of SNPs at at pH 2 (3A and 3B) and at pH 7.5 (5C) and contact angle of SNPs at pH 2.

FIGS. 6A, 6B, 6C, 6D-1, 6D-2, and 6D-3 are the optical microscope images of (6A) yeast, (6B) W/O/W Pickering emulsion at pH 4.3, (6C) W/O/W Pickering emulsion at pH 2, (6D-1 to 6D-3) change in the morphologies of W/O/W Pickering emulsion at 7.5 (scale bar: 20 µm).

FIGS. 7A and 7B. FIG. 7A is optical microscope images of Pickering emulsions at pH 2 after 2 weeks and B) actual vial images of Pickering emulsions before and after 2 weeks (scale bar: 50 µm).

FIGS. 8A and 8B. FIG. 8A shows the zeta-potential of shellac dispersion (5 mg/mL) at various pH (2-9), and FIG. 8B shows the diameter of CNCSHCA at various pH (2-9).

FIGS. 9A, 9B, 9C, and 9D. FIG. 9A shows control CO₂ release of yeast in varying pHs, FIG. 9B shows CO₂ release of yeast at various conditions at pH 2, FIG. 9C shows optical and fluorescence microscopy of yeast dyed with methylene blue to determine the viability at pH 2 (Scale bar: 50 µm), FIG. 9D shows optical and fluorescence microscopy of the partial coverage of yeast by CNCSHCA (dyed in methylene blue) to determine the viability at pH 2 (Scale bar: 50 µm).

FIGS. 10A and 10B are optical and fluorescence micrographs of encapsulation of yeast with (10A) CNCSHCA and (10B) TEM Micrographs of CNCSHCA. Scale bar; A: 50 µm, B: 500 nm.

FIGS. 11A and 11B. FIG. 11A shows confocal images of isolating the materials that compose the surface of the encapsulation; CNC, Shellac, oil, and merges capsule and FIG. 11B shows Z-sequences of optical slices (3D image stacks).

FIGS. 12A and 12B. FIG. 12A shows the determination of complex intensity through transverse scan and thickness of the coating and FIG. 12B shows secant along with the ark of the emulsion.

### DETAILED DESCRIPTION

The present disclosure relates to the use of ferulic acid (FA) functionalized cellulose nanocrystals (CNCs) (CNCFA), where the FA acts as an antioxidant and pH-responsive material for the preparation of “smart”, multiple Pickering emulsion containing yeast or probiotic cells with a modified plant-based emulsifier. Furthermore, the work intends to enhance the viability of yeast or probiotic cells during the emulsification process, storage, and intestinal delivery.

The present disclosure relates to the use of CNC-shellac (CNCSH) microcapsules in the presence of CaCl₂ (CNCSHCA). Shellac microcapsules maintain the survivability of S. cerevisiae or probiotic cells during exposure to simulated gastric and intestinal environments. The incorporation of CNC improved cells survival in gastric conditions.

As provided in this disclosure, once the modulation is established successfully, higher survivability against the severe acidic conditions in the stomach (pH 1.5-3) is observed. Hence, the Pickering emulsion can be delivered to the intestine, where the probiotics contained within the system are delivered due to the pH responsivity of the intestinal conditions. This system can be applied and scaled for food applications.

More importantly, the following advantages may be derived from the present disclosure:

(a) the CNCs are a highly applicable and sustainable emerging nanomaterial as novel emulsifiers for food applications.

(b) the functionalization of CNCs with FA is believed to render the CNCs as a substrate which facilitate the precipitation of the hydrophobic chemicals on the surface with high water dispersibility.

(c) the functionalization of CNCs with FA could act as an emulsifier for the oil and water phases to form highly stable emulsion.

(d) the functionalization of CNCs including FA in a non-toxic environment yield functionalized CNCs that can be greatly applicable in biomedical, and functional food industries.

Ferulic acid features both aromatic and antioxidant properties; the latter of these two properties is of interest regarding studies in mammalian toxicology leading to its desired use for CNC surface modifications and in vivo applications.

The complex of CNC and shellac with CaCl₂ is believed to render the CNCs as a reinforcement or filler in SH film at the water-oil interface, promoting better barrier to protect the active compounds. With the addition of CNC and divalent calcium cations, the network promoted by hydrogen bonding lowers the interfacial tension, which further promotes a stable network between shellac and CNC. The CNC is intercalated with the shellac and calcium ions at the water-oil interface forming an “impermeable” membrane resulting in a stable and spherical microcapsule.

FA and shellac are subjected to protonation in an environment where the pH of the solvent is lower than the pKa (such as a strong acid). Alternatively, FA and shellac will deprotonate in an environment where the pH of the solvent is greater than the pKa (such as a strong base).

Pickering emulsions stabilized by CNC-FA increase the structural stability for the encapsulation of active ingredients. The Pickering emulsions can have modifiability for processing conditions (in example: pH) such that applications like pH-sensitive drug delivery can be achieved.

Pickering emulsions made with food-grade particles possess low toxicity for in vivo application.

It is believed that multiple Pickering emulsions may be used; this type of emulsion is categorized by a dual layer stabilizer that is separated by opposite wettability such that one layer of the interface is, O/W and the other is W/O interface.

It is believed that Pickering emulsions with CNCSHCA may be used; this type of emulsion is prepared by anti-solvent method to precipitate and migrate to the water-oil interface. The anti-solvent effect drives the shellac to the water-oil interface to form a coating shell.

In one aspect, this concept describes the encapsulation of active probiotics within Pickering emulsions.

In one aspect, the emulsion is encapsulated by a modified CNC nanomaterial with a phenolic acid. This modification is made in attempts that the dissociation constant of the phenolic acid would cause pH-sensitive deprotonation, causing the emulsion to burst and releasing the probiotics.

In one aspect, the emulsion is encapsulated by a CNCSHCA complex. This modification is made such that the dissociation constant of the shellac would cause pH-sensitive deprotonation, causing the emulsion to burst and releasing the probiotics.

In one aspect, this release is designed to take place within the high pH environment of the intestine, and the probiotics will proliferate on the intestinal lumen.

In one aspect, application of this disclosure can be used to prepare various healthy microflora deliverable as a prophylactic or a treatment towards inflammation and infection of the intestine.

In one aspect, the successful release of probiotics in a high pH environment can gather methods to safely deliver appropriate treatments in vivo to benefit intestinal health.

In the examples below Trans-Ferulic acid (FA, MW=194.18, 99%), corn starch, baker’s yeast (saccharomyces cerevisiae) 1,1-diphenyl-2-picryl hydrazyl (DPPH), hydrochloric acid, sodium hydroxide, calcofluor white stain, methylene blue, and Nile red were purchased from Sigma-Aldrich (St. Louis, MO, U.S.A.). Shellac (Dewaxed Orange) was ordered from Inoxia Ltd (Cranleigh, United Kingdom). Cellulose nanocrystals (CNCs) were provided by CelluForce Inc. (Montreal, Canada). 100% denatured alcohol (with 5% isopropyl alcohol, 5% methyl alcohol, and ≤ 0.03% water) were purchased from Fisherbrand HistoPrepTM (Fisher, Ottawa, ON, Canada or Pittsburgh, PA) and Vegetable oil was purchased from a local market in Waterloo (ON, Canada).

### Example 1 - Preparation of CNCFA

1 g of CNCs and 60 mg of FA were dispersed in 0.8 mL and 2 mL of reagent alcohol, respectively. The FA solution was introduced into the CNC suspension then stirred for 24 h. The suspension was dried in the oven at 35° C. for 24 h. After drying, the suspension was re-dispersed in 200 mL of Milli-Q water. The instruments, UV-vis (Agilent 8453 UV-visible spectrophotometer, Santa Clara, CA, USA) and zetasizer (Zetasizer, Malvern, Nano ZS90, UK) were used to ensure that FA had adsorbed onto CNCs. For the UV test, all samples were placed in quartz cuvettes (Hellma Analytics) and the spectra were recorded at the wavelength range from 200 to 400 nm. They were filtered through ultrafiltration with a 0.1 µm filter membrane to remove unbounded FA. For measurement purposes regarding the characterization of FA, filtration was applied; however, FA will not be filtered for further experiments as free FA acts as a super antioxidant, rendering the removal of it as non-essential.

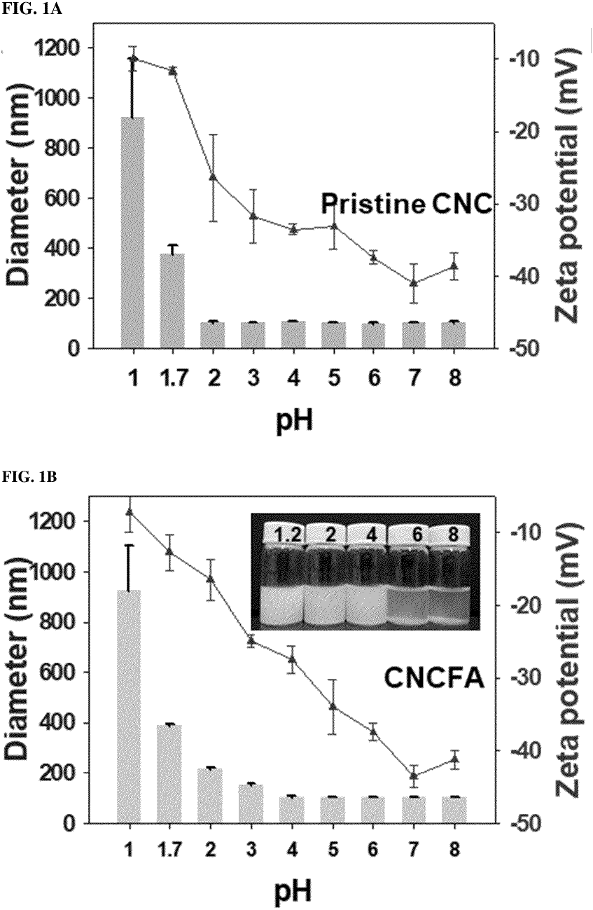

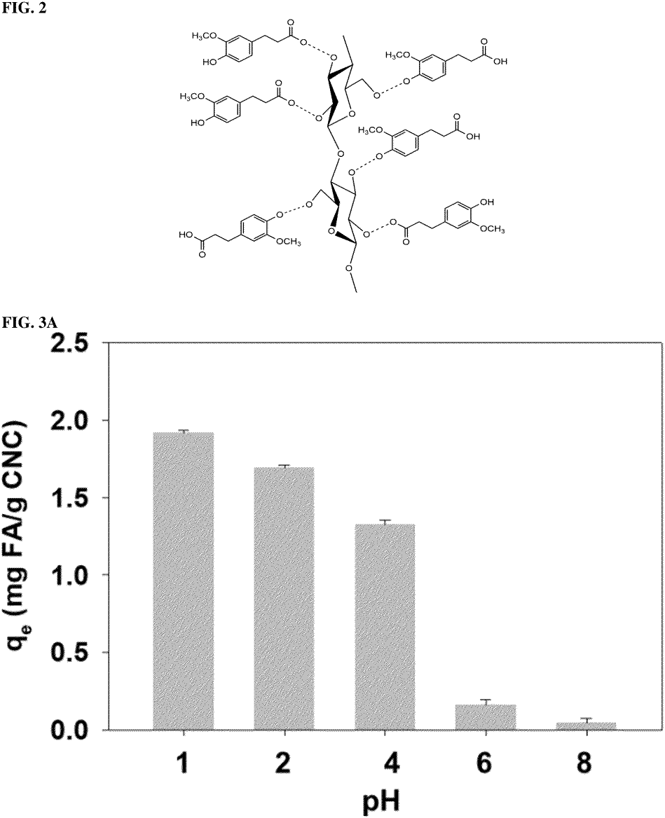

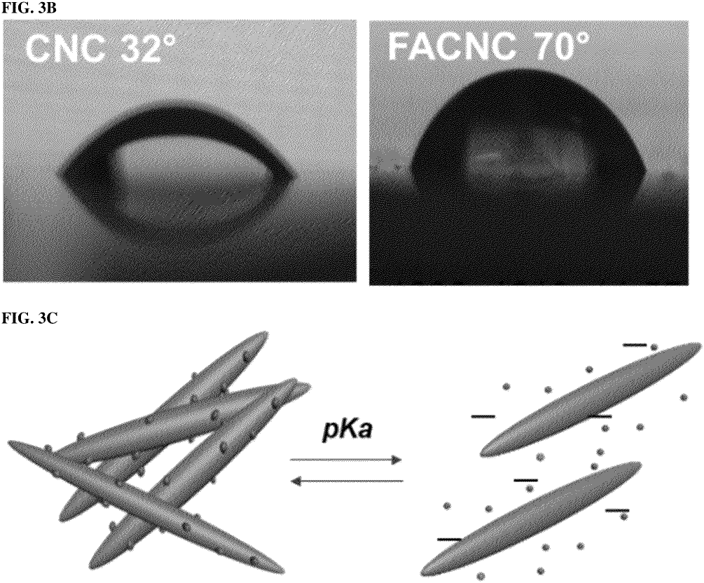

The adsorption equilibrium capacity (qₑ) was calculated according to the following equation (Dávila-Guzman et al., 2012):

$${q}_{e} = \frac{\left({C}_{0} - {C}_{e}\right) V}{m} \qquad \text{(1)}$$

where Cₒ and Cₑ are the initial FA and the equilibrium concentrations for 24 h, respectively; V is the solution volume in L; and m is the dry weight of CNC in g. The particle size and zeta-potential of CNC and CNCFA were conducted to elucidate the FA binding capabilities on the CNC. As shown in FIG. 1A, the CNC at all pH ranges from 1-9 has a negative zeta-potential for all pHs because of its sulphate ester groups on the surface. At a low pH of 1, the zeta potential of the CNC suspension increased to -10 mV. This is because the pKa of the sulfuric acid group is about 1.9. Therefore, the protonation of the sulfate group reduces the net charge below pKα. Interestingly, at pH 7.5. the zeta potential elevated to -40 mV. Qi et al. (2019) explained the reason that electron shielding occurred by adding Na⁺, resulting in the reduction of the zeta potential of CNC. However, at the same concentration, the effect of HCl on the zeta potential of a CNC suspension is greater than NaOH owing to the loss in the crystallinity and less reactive to ions upon the addition of —OH (Qi et al., 2019). On the other hand, in terms of the size of CNC dispersion at different pH levels, CNC aggregation yielding a particle size of around 900 nm as observed below the pKa value. It is formed because of attractive force induced by hydrogen bonding between CNC, which is more potent than the repulsive force. When pH was adjusted from 2 to 7.5, the sizes were constant at around 100 nm. In FIG. 1B, CNCFA dispersion at different pHs displayed a similar trend of size and zeta-potential, similar to the CNC dispersion. Still, with a slight difference, it showed a slightly larger size due to the adsorption FA onto the CNC via hydrogen bonding, hydrophobic interaction and lower zeta-potential values below the pKa value of FA. Therefore, when CNCFA is hypothetically subjected to a pKa greater than 4.5, the FA in CNCFA would detach from the CNC surface due to the repulsive forces from the negatively charge sulfate groups. Therefore, the changes in the adsorption of the phenolic acid may vary, depending on the pH, and salt concentration. To understand the strengthening mechanism of CNCFA nanoparticles, the binding mechanism of FA with CNC was elucidated. The interactions between FA and CNC suggested that the adsorption was associated with hydrogen bonding and hydrophobic interactions. Hydrogen bonding is considered the main driving force towards the formation of CNCFA due to the abundance of —OH groups on both the FA and CNC. The —COOH acid groups will be in a molecular form below the pKa value, thereby enhancing the hydrogen bonding and hydrophobic interaction (FIG. 2). But the hydrophobic interactions involve the benzene ring of FA and hydrophobic sites of CNC, (200) and the lattice plane (Bruel et al., 2019). FIG. 3A shows the adsorption capacity of FA to CNC at the different pH ranges. Below the pKa, the highest adsorption was observed, and the maximum adsorption number was 1.91 mg FA/g of CNC. On the other hand, at high pH, the adsorption capacity of FA was reduced to 0.03 mg FA/g of CNC because FA was ionized due to the dissociation of the carboxyl and hydroxyl groups. Many researchers have demonstrated that the adsorption capacity of ferulic acid on the different types of resin, such as zeolite (139 mg/g (Simon et al., 2015) and 203.2 mg/g (Thiel et al., 2013)), XAD7HP (63.6 mg/g) (Thiel et al., 2013), amberlite resin XAD16 (19 mg/g (Simon et al., 2015) and 133 mg/g (Dávila-Guzman et al., 2012)) as an inorganic adsorbent at different pHs and times. Moreover, there are few studies using dietary fibres (DFs), such as cellulose and xylan at digestive environment at pH 2 (stomach chyme), 4.5 (arithmetic), and 7 (ileum chyme). Each DFs were prepared at different pH levels using buffers. FA was dissolved in ethanol first and then transferred to the buffers with a 4% final ethanol concentration. The ratio of fixed FA (100 mg/L) and DFs were 1:50. At pH 2, the maximum adsorption occurred with 0.529 mg/100 mg of cellulose and 0.568 mg/100 mg of xylan. However, the author claimed that the FA adsorption onto DFs showed lower than the synthetic macroporous resin (11.7 mg/100 mg) from other studies because of a higher specific area of macroporous resins for between 400 and 1200 m²/g, comparing to lower specific area of microcrystalline cellulose, 2.39 m²/g (Costa et al., 2015). The contact angle of native cellulose and modified CNCFA were investigated using water contact measurement. FIG. 3B displays that the pristine CNC had a low contact angle of 32° because cellulose is interconnected by hydrogen bonding between —OH groups of CNC and water molecules to disperse, resulting in a relatively flat droplet (Trinh & Mekonnen, 2018). Whereas the contact angle of CNCFA increased to 70° with the carboxyl groups, leading to more hydrophobicity and increased in the wettability. The anticipated CNCFA at pH 2 and 7.5 are illustrated in FIG. 3C as a 3D model. Based on the results of several tests, FA was bound to CNC, and it became gel-like through hydrogen bonding reactions at acidic conditions. On the other hand, at pH 7.5, most FA was released due to its detachment from CNCs by repulsive force.

### Example 2 - DPPH Free Radical Scavenging Activity

The 2,2-diphenyl-1-picrylhydrazyl (DPPH) assay was conducted to measure the free radical scavenging activity of FA and CNCFA at pH 2 and 7.5 over an elapsed time. A DPPH solution (0.025 mg/ml) was prepared in methanol and covered with aluminum foil to avoid light exposure. 1.5 mL of an FA or CNCFA sample was added to 10 mL of the prepared DPPH solution in an aluminum foil covered vial and stirred. All absorbance was measured at 517 nm at different times (0, 5, 10, 15, 20, 25, and 30 min) using a Cary 100 Bio UV-Vis Spectrophotometer. Scavenging activity was calculated based on the equation:

$$\text{SA\%=} \frac{{\text{A}}_{\text{control}} - {\text{A}}_{\text{sample}}}{{\text{A}}_{\text{control}}} \times 100 \% \qquad \text{(2)}$$

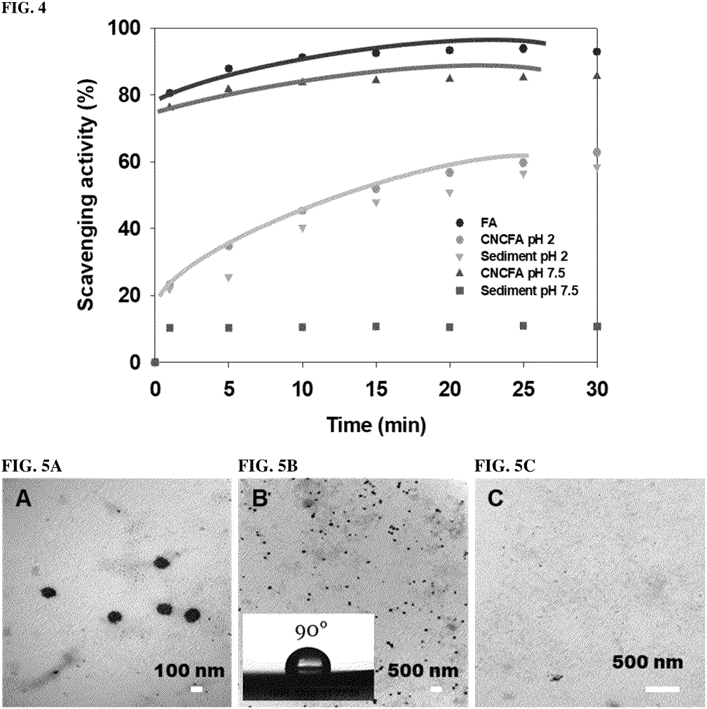

DPPH (2,2-diphenyl-1-picrylhydrazyl) is a purple compound that reacts rapidly with antioxidants to measure DPPH radical reduction hydrogen-donating ability through a colour change of purple to yellow. At this time, the electron-donating capability was measured at 517 nm and expressed as antioxidant power. The results of measuring the DPPH radical scavenging activity of FA, non-filtered CNCFA at pH 2 and 7.5 and filtered CNCFA at pH 2 and 7.5 are shown in FIG. 4. The FA Scavenging activity (% SA) was 99.1% at 30 min, showing the highest antioxidant activity. The antioxidant activity of FA is caused by the proton transfer from the phenolic and unsaturated carboxyl group to the DPPH radicals. Ferulic acid, which is a derivative of hydroxycinnamic acid, has higher antioxidant property than hydroxybenzoic acids, such as vanillic acid. FA is a powerful antioxidant that neutralizes free radicals and inhibits reactive oxygen species (ROS) or nitrogen to protect living organisms due to these functional groups. The phenoxy and hydroxy group of FA donate electrons to remove the free radicals. In addition, FA’s benzene ring stops the free radical reaction, and the carboxyl group also attacks the free radical site, preventing the premature oxidation of DNA and lipids as this group binds to the lipid bilayer (Srinivasan et al., 2007). This is due to the presence of (—CH═CH—) between the phenyl ring and a carboxyl group, inducing stronger hydrogen donating (Al Jitan et al., 2018). As it is known that the FA has a strong antioxidant effect, it showed an excellent antioxidation effect in 1 minute and almost 94% in 30 minutes. Surprisingly, CNCFA at pH 7.5 showed a similar effect to FA, but it had a little antioxidant effect after filtration. It suggests that most of the FA is attached to the CNCs. Regarding CNCFA at pH 2, almost 60% of the results were observed in 30 min both before and after the filtration.

### Example 3 - Preparation of SNPs

To replace synthetic emulsifiers with natural hydrophobic emulsifiers for W/O emulsion systems, starch nanoparticles were prepared using a nanoprecipitation method. 5 g of corn starch was dispersed in 100 mL of Milli-Q water in a round flask and heated at 90° C. to make a gelatinized solution. After 1 h, the temperature was set to 70° C. and then, 100 mL of ethanol was added dropwise to the gelatinized starch suspension with constant stirring (500 rpm) for 2 h. The suspensions were washed using ethanol at least 3 times by centrifugation (7000 rpm/ 7 min). The sediments were dried in the oven at 50° C. for 24 h and then ground to get fine powder. The white, fine powder obtained was used to determine some characteristics to utilize as a hydrophobic emulsifier. To confirm the morphology and mean size of the modified SNPs, TEM (TEM, Philips CM10 electron microscopy, acceleration voltage of 60 kV) and zetasizer (Zetasizer, Malvern, Nano ZS90, UK) were operated. To conduct wettability of SNPs in different pH of liquid, contact angle machine was used. In this study, to confirm the morphology and mean size of the modified SNPs, TEM and Zetasizer were operated. To conduct the wettability of SNPs in different pH of liquid, a contact angle machine was used. The results shown in FIGS. 5A and B, at pH 2 were consistent with the TEM images, as most particles were uniform with spherical particles at a diameter of 130 nm. Moreover, the PDI value was about 0.41, indicating that the SNPs distribution was homogenous (Table 1).

TABLE 1

Particle size and zeta-potential of SNPs at pH 2 and 7.5

| | pH 2 (A) | pH 7.5 (B) |
|---|---|---|
| Size (nm) | 138.6 ± 0.6 | 3760 ± 138 |
| PDI | 0.418 | 1 |
| Zeta-potential (mV) | -8.34 ± 0.8 | -12.5 ± 1.5 |

The value of contact angle was 90°. Theoretically, when the angle of the emulsifier is 90°, the stabilization energy of the emulsion is high; the particle would be in contact with two opposing phases without bias so that the surface shape of the droplet can be effectively maintained. However, some agglomeration was shown behind the particles due to the high energy of ultrasonication, which could cause the modified SNP to break down and swell. On the contrary, at pH 7.5 with high alkaline treatment, no spherical particles were shown. Only a heterogeneous matrix occurred (FIG. 5C). This is because alkaline materials such as urea or NaOH destroy the crystalline structure and increase solubility, whereas the minimum acid solubility of starch was observed at low pH.

### Example 4 - Preparation of W/O/W Multiple Pickering Emulsions

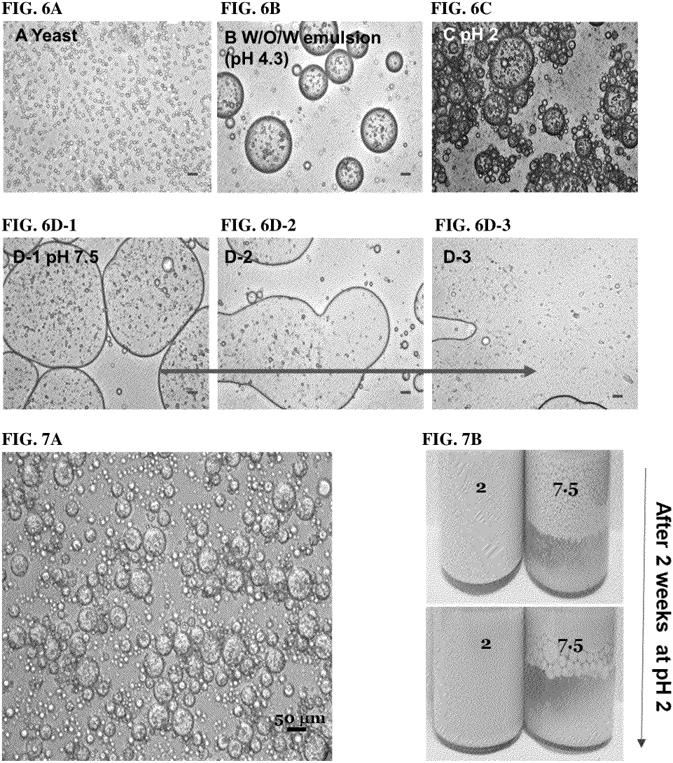

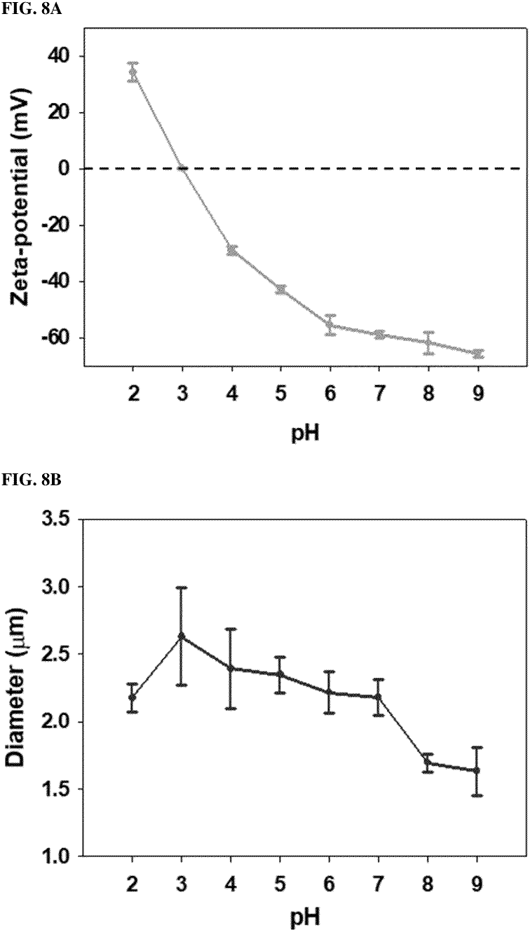

Multiple Pickering emulsions (W/O/W) were prepared by a two-step method. A W_{1,}_{yeast}/O emulsion (ratio 3:7) was the first step; the emulsion was stabilized by a hydrophobic emulsifier, which were the modified SNPs, through using a high-speed homogenizer (UltraTurrax T25 homogenizer, IKA, Germany) at 18,000 rpm for 3 mins. In the second emulsification step, W_{1,}_{yeast}/O/W_{2,}_{CNCFA} emulsions were obtained by the same high-speed homogenizer at 12,000 rpm for 2 mins. The ratio of the emulsion was 3:7 (W_{1,}_{yeast}/O)/W_{2,}_{CNCFA}); CNCFA nanoparticles, hydrophilic emulsifiers, were used. To distinguish the oil and water phases, 1 mg/mL of Nile red, which is a hydrophobic dye was used to stain the oil phase. The pH of the emulsions was adjusted to 2 and 7.5 to mimic gastric and intestinal pH, as well as to examine the viability of yeast. All samples were taken immediately right after by the microscope. The multiple Pickering emulsions (W/O/W) containing yeast at different pHs were observed through an optical microscope. The main factors in this study are the use of food-grade emulsifiers, CNCFA and SNPs, to absorb rapidly at the water-oil interface to reduce the interfacial tension and to release bioactive compounds (e.g., probiotics) in the intestine by deprotonation of the carboxyl group of FA. Besides, another vial factor of the multiple emulsion in the preparation step is to find the optimal rpm of the high-speed homogenizer. The higher driving force causes breakage of emulsion, followed by releasing active substance and starting coalescence between droplets resulting in phase separation. The purpose of this development is to ensure the safe passage of probiotics through the stomach where the strong gastric acid by encapsulating the probiotics in multiple emulsions. The freshly made W/O/W emulsions were transferred in each pH solution at 2 and 7.5 to see changes in morphologies. The change of oil droplets of W/O/W Pickering emulsions at different pH is shown in FIGS. 6A-D. These results were observed using an optical microscope utilizing bright-field microscopy. As a result of comparison with the original W/O/W Pickering emulsion (FIG. 6B), it was confirmed that the aggregation increased at pH 2 in FIG. 6C. Simultaneously, the oil droplets coalesced, burst, and disappeared as the pH increased beyond the CNCFA pKα when in a pH 7.5 environment (Figure 6D1-D3, time flow observation). It seems that this change is related to the zeta potential value between CNCs and FA. When CNCFA adsorbed on the oil droplets, the electrostatic repulsive force was lowered at low pH, resulting in aggregation; however, when the pH was increased to 8, the electrostatic repulsion also increased, causing the emulsion to become unstable due to the detachment of FA. 2 weeks stability test of the multiple Pickering emulsion at pH 2 and 7.5 was conducted, and the results are shown in FIG. 7. The emulsion was stable at pH 2 without phase separation, but separation started when the emulsion was transferred to a high pH solution from the start date. The optical microscopic images of Pickering emulsions at pH 2 confirmed that the particles of the emulsion after 2 weeks became slightly larger but showed that it continued to stabilize (FIG. 7A). The high stiffness and numerous hydroxyl groups on CNC acted as a nano-reinforcement and participated in the crosslinking of the hydrogen-bonded structure. Recently, there are many studies about phenolic or polyphenol-based emulsifiers for Pickering emulsions because they increase not only antioxidant effect but also improve stability such as gallic acid (GA) or tannic acid in zein nanoparticles. Interestingly, the phenolic nucleus of polyphenols could also be used as Pickering stabilizers at the oil-water interface. A complex of a polyphenol and whey protein demonstrated a significant improvement in the emulsion. Complex formation mechanisms could possibly be formed by hydrogen bonding, hydrophobic or electrostatic attraction between the protein at the interface with the polyphenol particles with opposite charges. Confocal images of the Pickering emulsion stabilized by the polyphenol particles and whey protein confirmed that the location of the complex at the water-oil interface. The emulsion showed high stability at pH 3 rather than at pH 7, due to the chemical decomposition of polyphenols at alkaline pH, resulting in the weaker complex (Zembyla et al., 2019). Table 2 shows the total number of yeasts in pH-treated Pickering emulsions calculated by standard broth dilution methods. The initial number of utilized yeasts was 10⁸, and the population of 3.14 × 10⁷ and 5.7 × 10⁶ CFU mg/mL has resulted in pH 7.5 and pH 2, respectively. The reduced yeast number obtained for lower pH value was interestingly different when seen under the microscope.

TABLE 2

The total number and survivability of yeasts in pH-treated Pickering emulsions

| Initial (10⁻⁴) | pH 2 | pH 7.5 |
|---|---|---|
| 10⁸ | 5.7 × 10⁶ | 3.14 × 10⁷ |
| Survivability (%) | 84.5 % | 93.71 % |

### Example 5 - Preparation of CNCSHCA

Shellac (5 mg/mL) was dissolved in 10 mL of denatured alcohol and redispersed in 190 mL of MQ water to achieve the desired concentration. CNCSH was prepared with 3 mL of shellac dispersion (5 mg/mL in 5% ethanol) and 2% CNC dispersion, and 0.8% CaCl₂ solution was added to CNCSH to produce CNCSHCA, which was used for future testing.

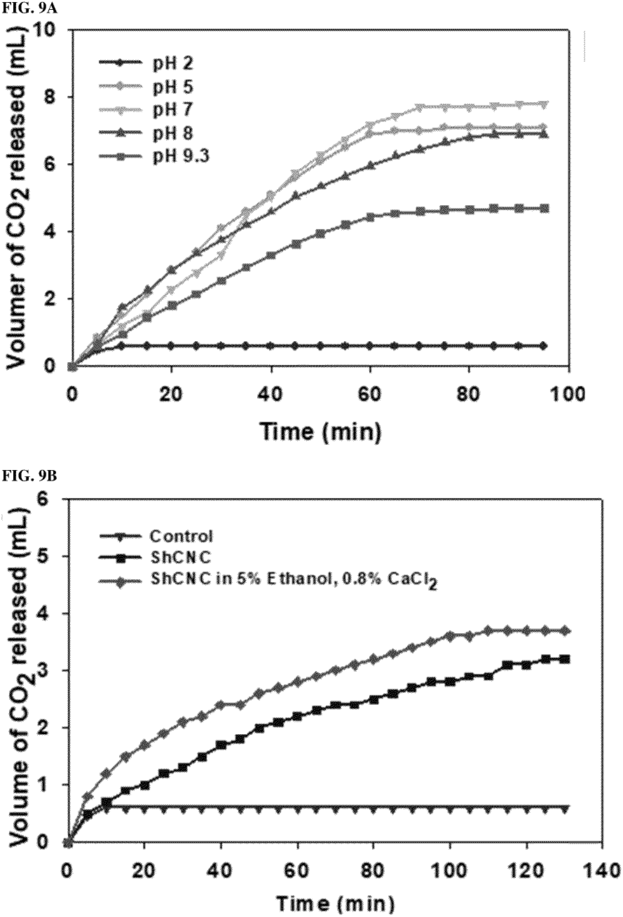

Shellac is known to be insoluble in water because of its many carboxyl groups (Penning, 1996). The protons of these carboxylic acids could be removed by dissolving shellac using alkaline solvents, such as sodium hydroxide and ammonia (Shellac has a pKα of 6.9-7.5) (Al-Obaidy et al., 2019). Additionally, shellac is entirely soluble in methanol, ethanol and partially soluble in ethyl acetate, chloroform and ether (Cagil, 2020). Since this study focuses on the development of microcapsules for food applications, ethanol at low concentrations, which is harmless to the human body was used. In addition to the solubility consideration, another crucial factor in selecting low concentration of ethanol is to ensure the viability of the probiotics or yeast used in the study. The 5 mg/mL in 5% ethanol was used as the optimum concentration to prepare shellac dispersions that was realized through anti-solvent precipitation. The zeta potential changed from -30 to -16 mV as the shellac concentration was increased, which is most likely caused by particle agglomeration that shielded the surface charges. FIG. 8A shows the zeta-potential trend of shellac dispersion (5 mg/ml in 5% denatured alcohol) from pH 2 to 9. As the pH approached the acidic conditions, the zeta-potential became less negative due to the addition of H⁺ ions. The influence of pH on the zeta-potential became more significant when the pH transitioned from 4 to 2. The protonation of the carboxylic groups in the shellac’s aleuritic acids and terpenic acids resulted in the zeta-potential approaching the isoelectric point (pI) at pH 3, which destabilized the shellac system (Spasojević et al., 2020) resulting in the precipitation and sedimentation of shellac particles.

In FIG. 8B, the size-distribution of the CNCSHCA complex between the pH of 2 to 9. In basic pH, shellac became soluble in a solution, as indicated by the reduction in the particle size and the light purple color of the dispersion. As the pH approached the acidic condition, the size of the CNCSHCA increased, reaching a maximum at pH 3, which correlates with the pI of the shellac dispersion. Shellac, being most unstable at this pH, would lead to the largest size of the complex, causing an increase in overall particle size. Despite this, there is no complete destabilization of the complex due to deprotonated -OH groups from the CNC.

### Example 6 - Yeast Survivability Test at Low pH

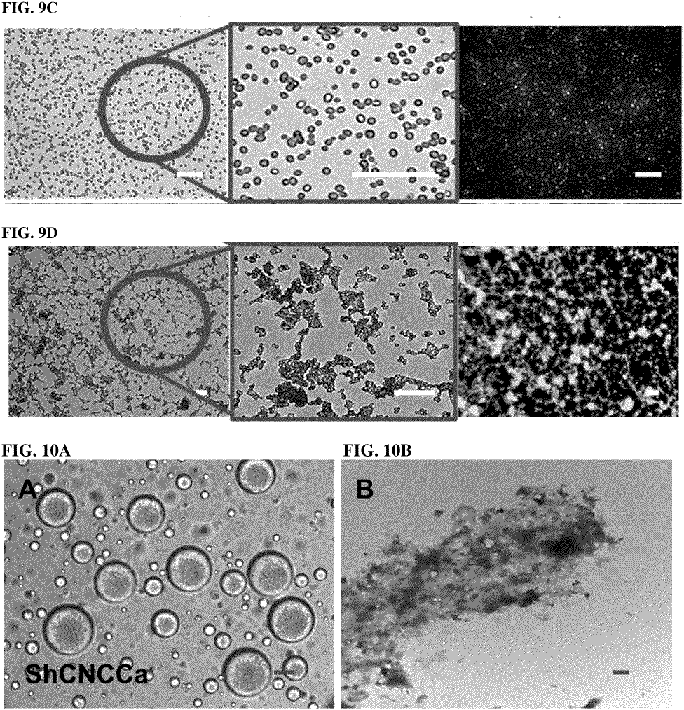

In order to evaluate the pH effect on the probiotics, we conducted the pH-controlled studies on the yeast by monitoring the CO₂ release as a function of time (FIG. 9A). S. cerevisiae produces CO₂ and ethanol as anerobic by-products in the fermentation when incubated with sugar at the optimum temperature and pH. We developed and utilized a method to assess the yeast activity by recording the CO₂ release using an in-house assembled apparatus. The hot plate was set to 40° C. to promote the metabolism of yeast in the presence of glucose at 400 rpm. It was conducted at four pHs (2, 5, 7.5 and 8) to measure the CO₂ production in order to assess the viability of the yeast. The test was performed by incubating 50 mg of Baker’s yeast with 50 mg D-glucose in 5 mL MQ-water. Three trials were performed at each pH value and an averaged value was recorded.

At pH 2, the amounts of CO₂ released was 0.6 ml, indicating that the yeast cells had lysed. At pH of 5, 7.5, and 8, the CO₂ release did not deviate. However, at pH 9.3, the total amount of CO₂ released was significantly decrease because yeast can survive up to pH 7.5 (Reethu Narayanan & Ch, 2012). At pH 7.5, yeast produced the most amount of CO₂, signifying that the optimal condition for yeast viability was around 5 to 7.5, and it decreased when the environment became basic. Comparing the CO₂ release tests (FIG. 9B) of the control, CNCSH and CNCSHCA at pH 2, approximately 5 times more CO₂ was released due to the coating of CNCSHCA on the yeast cells that preserved the yeasts in unfavorable pH conditions. The partial coverage of yeast was possible due to the followings: (1) CNC acting as a reinforcement in the polymer matrix (Obradovic et al., 2017) and Ca²⁺ as a bridging ion (Luo et al., 2016) to cross-link the carboxyl groups of shellac, (2) zeta-potential of yeast at pH 2 was positive as it is below the pI of the yeast cell wall protein (Narong & James, 2006); hence it could interact with the negative charges via electrostatic interaction to form the shellac complex (Rogowska et al., 2018). Additionally, yeast was dyed with methylene blue so as to enable us to check its viability under the microscope and compare it with CO₂ test results. CaCl₂ was added to the yeast dispersion to improve its stability, while Ca²⁺ ions are known to promote yeast growth (Cui et al., 2009). Thus, the CO₂ test was conducted to examine the impact of the encapsulation strategy on the viability of yeast cells. Kwolek-Mirek (2014) states that living cells enzymatically reduce the dye or pump methylene blue out of the cell. Hence live yeast cells do not stain (FIG. 9C), while dead cells are stained blue (Kwolek-Mirek & Zadrag-Tecza, 2014). Therefore, the live and dead yeast could be clearly distinguished under different pH conditions. Moreover, dead cells that are stained with methylene blue emit red fluorescence under a fluorescence microscope. From the magnified image of FIG. 9C, many dead yeast cells were observed when they were incubated at pH of 2. However, once CNCSHCA was added to the yeast, partial coverage on yeast occurred (FIG. 9D) that was observed as green fluoresce shellac, and orange fluoresce MB dyed shellac coated yeast from the fluorescence image. Interestingly, we found an important observation that the shellac is auto-fluorescence (Bellan et al., 2012).

### Example 7 - Preparation of W/O Pickering Emulsions

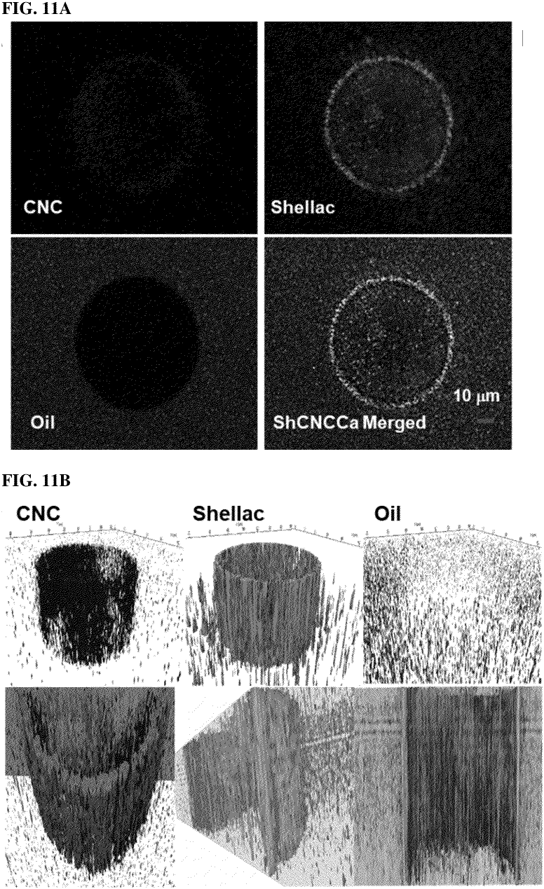

A W/O emulsion (ratio 4:6) was prepared using Pickering emulsification technique. Specifically, 30 mg yeast was dispersed in water to hydrate, and 100 µL methylene blue (10 mg/mL) was added. The excess dye was removed using a centrifuge at 3,000 rpm for 2 min. Then, 3 mL of 5% shellac dispersion was added to the dyed yeast and mixed with vegetable oil using a high-speed homogenizer (Ultra Turrax T25 homogenizer, IKA, Germany) at 9,500 rpm for 3 min, followed by the addition of 2% calcofluor white dyed CNC (CNC-CW) and 0.8% CaCl₂ solution to improve the emulsion stability. To distinguish between the oil and water phase, 1 mg/mL of hydrophobic Nile red and 10 mg/mL of hydrophilic CW were used. To precisely detect the CNC-CW, unbound CW was removed by ultrafiltration with a 0.1 µm filter. The emulsions’ pHs were adjusted to 2 or 7.5 to mimic the gastric and intestinal pH conditions and the yeast viability was assessed. when CaCl₂ was incorporated in the emulsion prepared with CNCSH, uniform spherical particles were observed with a significant increase in the encapsulated yeasts (FIG. 10A). This demonstrated that calcium ions play a major role in the formation of a strong cross-linking and stable network between shellac and CNC (FIG. 10B). This resulted in the improved retention of yeast cells as shown by the average number of yeast particles encapsulated per droplet. With only the shellac complex, the average number of yeast particles recorded per droplet was 20.86. With calcium ions to screen the charge and CNC for hydrogen bonding interaction with shellac, robust microcapsules cannot be formed to encapsulate and preserve the high number of yeast particles. However, with the CNCSHCA complex, where the additional interactions from CNC and calcium ions produce robust microcapsules, where the average number of yeast particles encapsulated per droplet increased to 125.65, representing a 602.35% increase in yeast retention.

### Example 8 - Preparation of W/O Pickering Emulsions

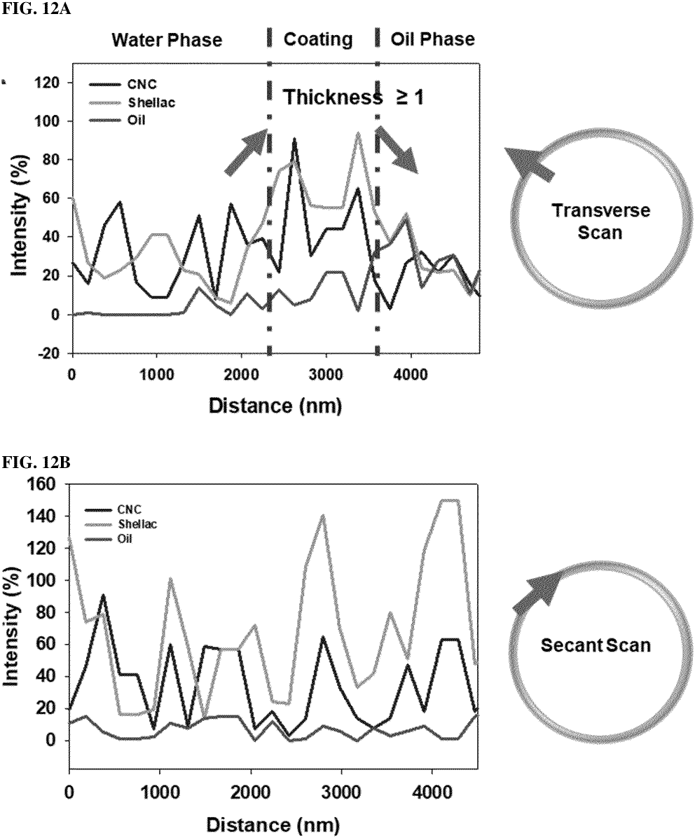

The morphologies of the yeast, CNCSH, CNCSHCA complex and yeast encapsulated Pickering emulsions at pH 2 and 8 were analyzed using a transmission electron microscope, Philips CM10 (TEM), optical, fluorescence (Nikon Eclipse Ti-S, Nikon Instruments Inc., USA), and confocal microscope (Zeiss LSM 510 Meta Laser Scanning Confocal Microscope (CLSM). To calculate the retention of yeast in different microcapsules, the number of yeast particles was analyzed using the ImageJ, and the average of all counts was determined from the optical micrographs. To confirm the CNCSHCA complex formation in the Pickering emulsion, CNC and oil were stained using a Calcofluor white and Nile Red, respectively, while shellac possessed a natural auto-fluorescence. The confocal 3D stacked images obtained using the CLSM provided information on the distribution of the CNC, shellac and oil in the Pickering emulsion. FIG. 11A shows the 2D image that confirmed that both CNC and shellac formed complexes at the water-oil interface. FIG. 11B shows the cross-section of the emulsion droplet, where the oil phase segregated from the water phase, and the CNCSHCA complex was located at the water-oil interface. The CNCSHCA complex lowered the interfacial tension of the water-oil that yielded an emulsion that was stabilized against flocculation and coalescence. To provide further evidence on the structure of the microcapsule, the sample was scanned using the imaging software in two configurations, and the direction of the scan is indicated by the arrow in FIGS. 12A and 12B: (1) transverse scan through the microcapsule and (2) secant scan along the circumference of the microcapsule. In FIG. 12A, the three distinct phases within the microcapsule could be determined, where the water phase within the capsule (between 0-2300 nm), coating (2300-3600 nm), and the oil phase (>3600 nm) were identified. In the water phase, the intensity of the fluorescence from the oil was close to zero, while there was detectable fluorescence from both the CNC and shellac. The intensity of the coating layer consisted mainly of CNC and shellac, suggesting that it comprised mainly of these two major components. Both the green and blue fluorescence intensities were detectable in the water phase, confirming that the shellac precipitated when the denatured methanol evaporated, and the precipitated shellac then complexed with the CNC and calcium at the water-oil interface. However, near the coating layer, the green and blue peaks were significantly larger, indicating the presence of both higher amounts of shellac and CNC. The coating thickness was estimated to be about 1- 1.3 microns using the software. In the oil phase region of the coating, the intensity of CNC and shellac began to decrease, and the intensity from the oil peak began to rise. In FIG. 12B, a secant scan was conducted along with the circumference/surface of the microcapsule as indicated by the red arrow. Green and blue peaks appeared together, while the red intensities were close to zero, indicating that the oil was absent at the surface of the coating. The evidence from confocal microscope analyses suggested that the emulsion surface was composed of shellac, CNC and CaCl₂.

What is claimed is:

1. A method for producing ferulic acid (FA) and functionalized highly stable cellulose nanocrystals (CNCs)-CNCFA.

2. A method for producing modified starch nanoparticles (mSNP).

3. Two optimized methods for producing a W/O emulsion stabilized by mSNP for yeast (or a probiotic derivative thereof) encapsulation.

4. A method for DPPH assay to observe the scavenging activity of free radicals; to aid in interpretation of CNCFA multiple Pickering emulsion stability.

5. A method for producing a W/O/W multiple Pickering emulsion stabilized by CNCFA for yeast (or a probiotic derivative thereof) encapsulation.

6. The use of mSNP as a hydrophobic stabilizer used to keep water and oil phases emulsified in the W/O phase of a yeast (or a probiotic derivative thereof) encapsulated multiple Pickering emulsion as described herein.

7. A method for CO₂ Production on different pH by yeast.

8. The use of CNCFA as a hydrophilic stabilizer used to keep water and oil phases emulsified in the W/O/W phase of a yeast (or a probiotic derivative thereof) encapsulated multiple Pickering emulsion as described herein.

9. The nature for CNCFA and mSNP based multiple Pickering emulsion to be used for probiotic delivery.

10. The nature for CNCFA to be pH responsive, elucidating the release and delivery of yeast (or a probiotic derivative thereof) due to coalescence of the multiple Pickering emulsion as described herein.

11. A method for producing Shellac (SH) and cellulose nanocrystals (CNC) complex with CaCl₂-CNCSHCA.

12. An anti-solvent method for producing encapsulated yeast (or a probiotic derivative thereof) microcapsules.

13. A method for CO₂ gas production to observe yeast (or a probiotic derivative thereof) survivability at various pH conditions.

14. A method for producing a W/O Pickering emulsion stability by CNCSHCA for yeast (or a probiotic derivative thereof).

15. The use of CNC and CaCl₂ as reinforcement and crosslinker agent to form a rigid complex shellac coating in the W/O phase of a yeast (or a probiotic derivative thereof) encapsulated.

16. The rigid and controlled enteric coating for CNCSHCA to be pH responsive, elucidating the release and delivery of yeast (or a probiotic derivative thereof) due to break down of the multiple Pickering emulsion as described herein.
